(19) United States
(12) Patent Application Publication
(10) Pub. No.: US 2005/0049551 A1
(43) Pub. Date: Mar. 3, 2005

(54) Administering device with multiple-chamber ampoule and mixing block

(76) Inventor: Fritz Kirchhofer, Sumiswald (CH)

Correspondence Address: David E. Bruhn, Esq.;DORSEY & WHITNEY LLP, Intellectual Property Department, 50 South Sixth Street, Suite 1500, Minneapolis, MN, 55402-1498, US

(21) Appl. No.: 10/927,624

(22) Filed: Aug. 26, 2004

(30) Foreign Application Priority Data
Sep. 3, 2003 (DE) DE 103 40 585.2

(51) Int. Cl. A61M 37/00

(52) U.S. Cl. 604/82; 604/232

(57) ABSTRACT

A device for administering an injectable product from a multiple-chamber ampoule including a first casing part and a second casing part into which the ampoule can be inserted, wherein the second casing part can be moved relative to the first casing part in order to mix the injectable product prior to administering it. In one embodiment, a holder releaseably holds the second casing part in a fixed position relative to the first casing part.

### BACKGROUND

The present invention relates to devices and methods for administering, dispensing or delivering substances. More particularly, it relates to an administering device for administering, injecting or dispensing an injectable product from a multiple-chamber ampoule. The application claims the priority of German patent application No. 103 40 585.2, filed on Sep. 3, 2003 with the German Patent and Trademark Office.

Some medical, therapeutic or pharmaceutical active agents cannot be administered in solid form, but also cannot be stored over a long period of time in a dissolved state as an injectable fluid product. Such active agents may be provided in multiple-chamber ampoules which comprise a first chamber for a first product component, e.g., a solid product component, and at least a second chamber for a second product component such as a solvent. The first product component may be an active agent, or vice versa. The two chambers are separated from each other by a stopper which can be moved axially with respect to the ampoule, and the second chamber is sealed off by a second stopper. One example of such an injection device and method is disclosed in U.S. Pat. No. 5,728,075, the disclosure and teachings of which are incorporated herein by reference. Other examples of multi-chamber injection devices and ampoules are known to those skilled in the art as well.

Typically, the active agent is not mixed until shortly before administering. It is then mixed by axially shifting the two stoppers in the ampoule, such that the solvent enters the first chamber via a redirecting channel and dissolves the solid active agent. The active agent is then available as an injectable fluid product. The multiple-chamber ampoules are generally stored separately from an administering device, in order to prevent them being inadvertently mixed or only partially mixed by the administering device due to interference or jolts.

Known devices for administering the injectable product from the multiple-chamber ampoule comprise a first casing part and a second casing part which can be removed from the first casing part. For administering, the multiple-chamber ampoule—which is stored separately—is inserted into the second casing part, and for mixing the injectable product is attached to the first casing part and moved relative to it. By moving the casing parts relative to each other, such as, for example, sliding them together or rotating them together, the stoppers in the multiple-chamber ampoule are shifted axially in the multiple-chamber ampoule by a drive means of the administering device, whereby the two product components are mixed. After mixing, the administering device is ready for administering the fluid product. In this method, a number of individually provided components, which have to be assembled, and a multitude of preparatory steps are necessary for preparing to administer, which makes administering an active agent awkward and tedious.

### SUMMARY

It is an object of the present invention to provide a device for administering an injectable product from a multiple-chamber ampoule which facilitates and accelerates the administering procedure and enables the preparation for administering to be checked.

The object is addressed in accordance with the invention by providing an administering device for administering an injectable product from a multiple-chamber ampoule comprising a first casing part and a second casing part into which the multiple-chamber ampoule can be inserted and which can be moved relative to the first casing part in order to mix the injectable product, comprising a holding means which fixedly holds the second casing part detachably on the first casing part in a fixed position, before the injectable product is mixed in the multiple-chamber ampoule.

Accordingly, in one embodiment, an administering device for administering an injectable product from a multiple-chamber ampoule comprises a first casing part and a second casing part. The casing parts can be formed as sleeves, wherein the sleeve parts exhibit a fitting shape such that they can be slid one over the other. Preferably, the second casing part can be shifted into the first casing part, wherein the outer circumferential area of the second casing part abuts the inner circumferential area of the first casing part. The multiple-chamber ampoule can be inserted into the second casing part. For this purpose, the second casing part is open at one end and constricted at the other end such that the multiple-chamber ampoule, after it has been inserted through the open end, pushes against the constriction. An injection needle with a fluid connection to the multiple-chamber ampoule can be attached to the constricted end of the second casing part. The multiple-chamber ampoule advantageously sits in the second casing part with zero clearance. In order to mix the injectable product, the second casing part is attached to the first casing part and moved relative to the first casing part. The second casing part can, for example, be rotated or shifted with respect to the first casing part. The second casing part is preferably inserted, via its open end, into an open end of the first casing part in an exact fit, and can then be moved into the first casing part via a thread or guiding grooves between the inner area of the first casing part and the outer area of the second casing part. When the second casing part is moved in, a drive means acts on a stopper of the multiple-chamber ampoule, in order to mix the injectable product.

In accordance with the present invention, the administering device comprises a holding means which fixedly attaches the second casing part detachably to the first casing part, i.e., the second casing part can be detached from the first casing part, but is held in a fixed position with respect to the first casing part by the holding means. In this fixed position, the administering device is in a starting position before mixing the injectable product within the multiple-chamber ampoule. In the fixed, assembled position of the first and second casing part, the multiple-chamber ampoule is already inserted into the second casing part. Furthermore, in the fixed position of the holding means, a drive means of the administering device can already be engaging with a piston or come to rest a small distance from it. In the fixed starting position for mixing the product, however, the stopper has not yet been moved within the multiple-chamber ampoule.

In one embodiment, the present invention comprises a device for administering an injectable product from a multiple-chamber ampoule including a first casing part and a second casing part into which the ampoule can be inserted, wherein the second casing part can be moved relative to the first casing part in order to mix the injectable product prior to administering it. In one embodiment, a holder releaseably holds the second casing part in a fixed position relative to the first casing part.

With the aid of the present invention, it is possible to provide an administering device for administering a product from a multiple-chamber ampoule in an assembled state for the user, without there being a danger of inadvertently or prematurely mixing the product components in the multiple-chamber ampoule. In its assembled state with a multiple-chamber ampoule already inserted, the administering device can be stored over a long period of time, since the individual casing parts are arranged in a fixed position with respect to each other by the holding means and are held in said position. A user does not have to firstly assemble the individual parts of the administering device before administering, and prepare or perform mixing by attaching the ampoule to the administering device.

In one embodiment of the present invention, the second casing part can therefore be released from the fixed position in the holding means and moved relative to the first casing part into a mixing position. For this purpose, the second casing part is moved out of the hold of the holding means into the first casing part such that the stoppers in the multiple-chamber ampoule are shifted by the drive means of the administering device. The holding means is preferably formed by a latching means between the first casing part and the second casing part, which exhibits a latching position corresponding to the fixed position of the second casing part before mixing. The latching means advantageously exhibits a second latching position in which the fluid product is completely mixed. By latching into the second latching position in the mixing position, a user can recognize that the mixing procedure has been performed. The second casing part can, for example, be released from the fixed position manually or with the aid of a trigger which can be manually or automatically operated. If released manually, a resistance of the holding means is overcome by muscular strength such that the second casing part is released from the fixed position. In the case of a manual trigger, a triggering button can be pushed or also rotated into a triggering position, in order to then move the second casing part out of the fixed position by hand. A biasing element can be used to provide an automatic trigger which is held in a biased position by a fixing element and transfers the biasing force onto the second casing part when the fixing element is released.

In one embodiment, a protrusion can be provided as a latching means on the outer circumference of the second casing part and engages with a groove on an inner circumferential area of the first casing part. The protrusion can be formed by individual elevations arranged in the circumferential direction or by an annular elevation. Grooves for the individual elevations are correspondingly arranged on the second casing part. By the protrusion latching into the groove, the second casing part is fixed with respect to the first casing part. In order to release this fixed position, the second casing part is shifted, e.g., by muscular strength, against the resistance force exerted on the projection by the edge of the groove. The projections are particularly advantageously made of a rubber-like material, such that they are elastically compressed in order to be inserted into the groove and relax when latched into the groove. For the mixing position, a second groove can be provided which is arranged at a distance from the first groove which corresponds to the distance which the stoppers in the multiple-chamber ampoule have to travel during the mixing procedure.

It is also possible to provide the holding means by a protrusion on the second casing part, said protrusion pushing against a stop, such as the facing edge of the first casing part, when the second casing part is inserted into the first casing part. A protrusion can in principle also be provided on the first casing part which pushes against a stop on the second casing part.

Other embodiments can be provided. For example, a latching means can be used which comprises a number of consecutive teeth on a casing part which engage with complementary teeth on a second casing part. Such latching means or the like can ensure that the first casing part can only be moved in one direction, into the second casing part in order to mix the fluid product. Such a latching means can prevent the first and second casing part from being re-detached from each other. Once the casing parts have been moved further, into the mixing position, it is then possible to release the latching connection between the first and second casing part using corresponding guiding means, such that the casing parts can be detached from each other in order to be able to insert a new, unmixed multiple-chamber ampoule into the second casing part.

It may be seen from the examples described that a holding means or latching means can be provided by different mechanisms as long as the first and second casing part are held or latched in a position in which the stoppers of the multiple-chamber ampoule are not moved.

In a preferred embodiment of the present invention, a safety indicator is provided which indicates a movement of the second casing part about or out of the fixed position. This enables a user to determine whether the administering device has been handled properly, such that it is certain that the stoppers in the multiple-chamber ampoule have not yet been moved and the active agent has not taken any damage. A tearing adhesive tape can serve as the safety indicator, attached over the first and second casing part in a bordering region. A movement of the first casing part with respect to the second casing part causes a change in the adhesive tape or even causes the tape to tear, from which it becomes clear that the second casing part was not arranged in the fixed position with respect to the first casing part at any time. There is therefore the possibility that the multiple-chamber ampoule has been damaged or that the product components in the ampoule have already been partially mixed. A safety indicator could also be provided by a coloration in which different color ranges are visible in the event of relative movement or in which relative movement generates a change in color.

### BRIEF DESCRIPTION OF THE DRAWINGS

FIG. 1 is a longitudinal section through an administering device in a fixed position;

FIG. 2 is a detail of a holding means in accordance with the invention; and

FIG. 3 is a longitudinal section through an administering device in a mixing position.

### DETAILED DESCRIPTION

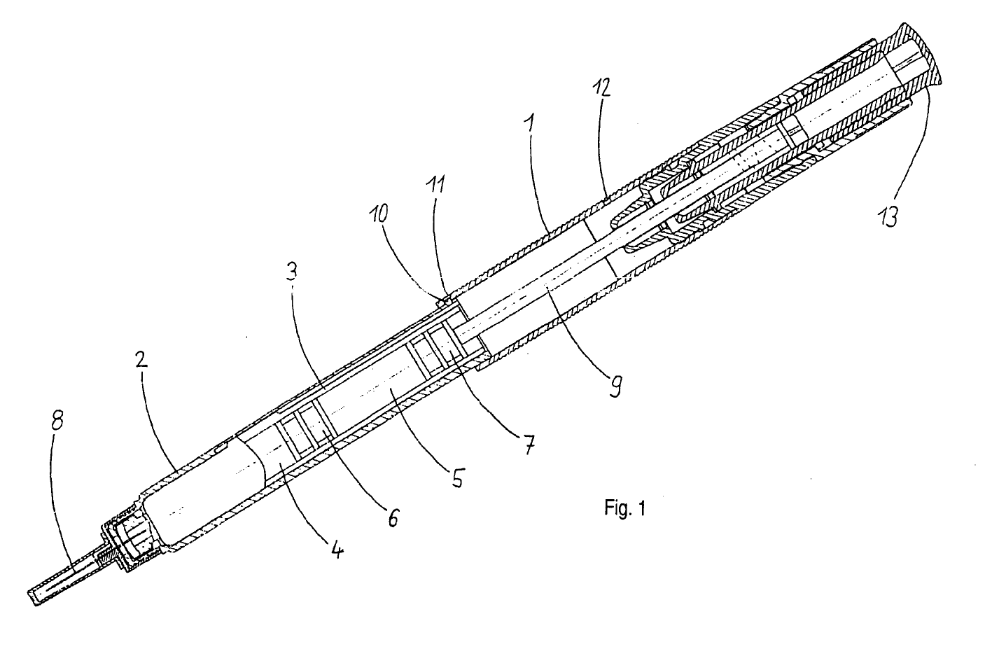

FIG. 1 shows an administering device comprising a first casing part 1, a second casing part 2 and a multiple-chamber ampoule in the form of a bicameral ampoule 3. The bicameral ampoule 3 comprises a first chamber 4, a second chamber 5, a first stopper 6 which separates the first and second chamber, and a second stopper 7 which seals off the second chamber 5. The second casing part 2 is formed in the shape of a sleeve and comprises an injection needle 8 at a front, constricted end, said injection needle having a fluid connection to the second chamber 5. The second casing part 2 is open at the other, opposite end, such that the bicameral ampoule 3 can be inserted into the second casing part 2. An administering mechanism comprising a drive member 9 in the form of a piston rod is accommodated in the first casing part 1.

FIG. 1 shows the administering device in a first position in which the second casing part 2 together with the bicameral ampoule 3 is attached to the first casing part 1. The second casing part 2 connected to the first casing part 1 in a fixed position by a holding means in accordance with the invention, but can be released from the holding means.

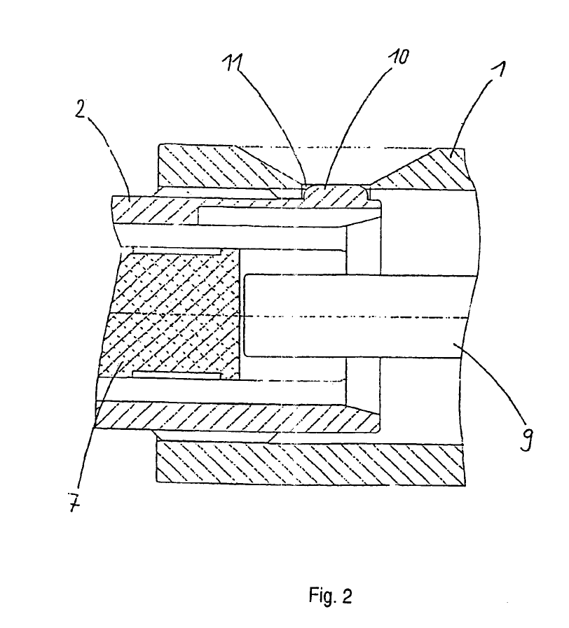

As shown in detail in FIG. 2, the holding means comprises a protrusion 10 on the outer circumferential area of the second casing part 2 and a groove 11 on the inner circumferential area of the first casing part 1. In the fixed position, the protrusion 10 engages with the groove 11. The position at which the protrusion 10 is provided on the second casing part 2 and the position at which the groove 11 is provided on the first casing part 1 are adjusted to each other such that when the groove 11 engages with the protrusion 10, the drive member 9 almost touches the stopper 7 and does not yet exert a force on the stopper 7.

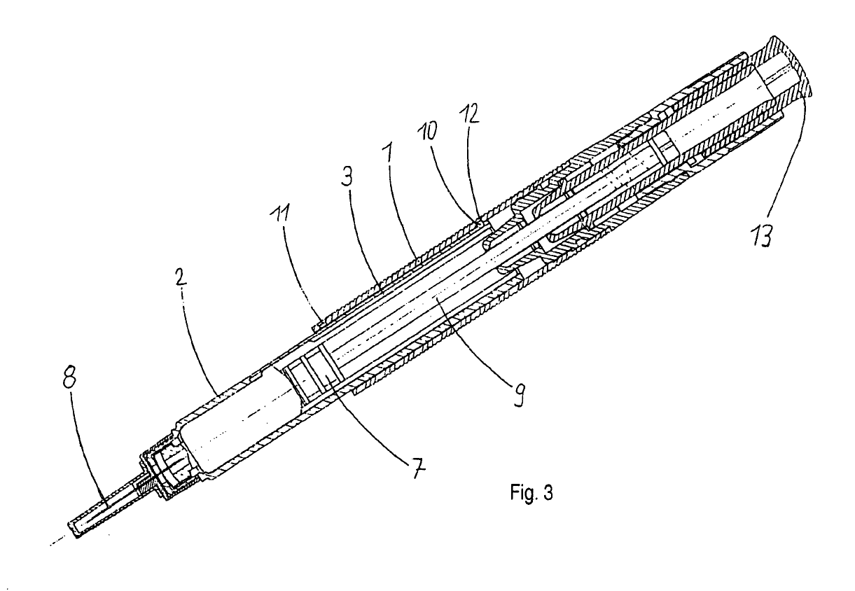

FIG. 3 shows the administering device shown in FIG. 1, in a second position in which the fluid product in the bicameral ampoule 3 is mixed, i.e., in a mixing position. In order to mix the injectable product in the bicameral ampoule 3, the second casing part 2 is moved into the first casing part 1. If a safety indicator as has been described above is provided over the transition region between the first and second casing parts 1 and 2, this is removed beforehand for mixing, as applicable, e.g., an adhesive tape is removed from this region of the administering device. The casing part 2 is released from the fixed position of the holding means by being slid or rotated, wherein the protrusion 10 is guided out of the groove 11, against a resistance.

The second casing part 2 is moved with respect to and into the first casing part 1 until the drive member 9 has shifted the stopper 7 axially with respect to the bicameral ampoule far enough that the fluid product has been adequately mixed. In this mixing position, a groove 12 in the inner circumferential area of the first casing part 1 is situated opposite the protrusion 10 and the protrusion 10 of the second casing part 2 engages with said groove 12. The mixing position is characterized by this engagement. Correspondingly, the distance between the first groove 11 and the second groove 12 is selected such that it corresponds to the path distance of the stopper 7 necessary for adequate or complete mixing. In the mixing position, the administering device is ready to administer the injectable product. The injectable product can, for example, be administered by activating a button 13 which triggers an administering mechanism (which may be of any suitable type, including those known to those skilled in the art), such that the fluid product is delivered through the injection needle 8.

In the foregoing description, embodiments of the present invention, including preferred embodiments, have been presented for the purpose of illustration and description. They are not intended to be exhaustive or to limit the invention to the precise forms disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for administering an injectable product from an ampoule, said device comprising a first casing part and a second casing part into which the ampoule can be inserted and which can be moved relative to said first casing part in order to mix the injectable product, and a holding means which fixedly holds the second casing part detachably relative to the first casing part in a fixed position before the injectable product is mixed.

2. The administering device as set forth in claim 1, wherein said holding means comprises a protrusion on a circumferential area of one of the first or second casing part and a stop on the other casing part.

3. The administering device as set forth in claim 1, wherein the holding means is formed by latching means between the first casing part and the second casing part, said latching means providing a latched position as a fixed position.

4. The administering device as set forth in claim 1, wherein the second casing part can be released from the fixed position of the holding means manually or by means of a trigger.

5. The administering device as set forth in claim 1, wherein a safety indicator is provided to indicate a movement of the second casing part from the fixed position in the holding means.

6. The administering device as set forth in claim 5, wherein said safety indicator comprises a tearable member.

7. A device for administering an injectable product from a multiple-chamber ampoule comprising a first casing part, a second casing part into which the ampoule can be inserted, wherein the second casing part can be moved relative to the first casing part in order to mix the injectable product prior to administering it, and a holder which releaseably holds the second casing part in a fixed position relative to the first casing part.
